(19) United States
(12) Patent Application Publication
(10) Pub. No.: US 2005/0101838 A1
(43) Pub. Date: May 12, 2005

(54) Endoscope cover

(76) Inventors: Philip L. Camillocci, Stuart, FL (US); Craig E. Stickles, Lyles, TN (US)

Correspondence Address: Patrick S. Yoder, FLETCHER YODER, P.O. Box 692289, Houston, TX, 77269-2289, US

(21) Appl. No.: 10/706,197

(22) Filed: Nov. 12, 2003

(51) Int. Cl. A61B 1/00

(52) U.S. Cl. 600/125; 600/127

(57) ABSTRACT

The present invention provides a technique for a covering and identifying an endoscope. In one embodiment, a cover may be placed onto the probe portion of an endoscope. Advantageously, the cover may protect the probe portion against inadvertent damage. Additionally, the cover may comprise an indicium indicative of a status of the endoscope or endoscope components, such as the probe portion. For example, the indicium may comprise colors, such as red and green, respectively assigned to indicate a sterile or contaminated condition of the endoscope or endoscope components.

### BACKGROUND OF THE INVENTION

The present invention relates generally to the field of endoscopes. More particularly, the invention relates to a technique for identifying and protecting a probe portion of an endoscope.

To diagnose a patient, it may become necessary for a medical professional, such as a physician, to determine the condition of a patient's anatomy or organs. Healthy tissue may present a different appearance in comparison to diseased or abnormal tissue. Moreover, in certain other instances, it may become necessary for a physician to retrieve or biopsy a tissue specimen for further testing. Exploratory surgery and biopsies are often relatively invasive procedures. For instance, such traditional procedures may require relatively large incisions and anesthesia, which are typically to be avoided. Moreover, many such procedures may require general anesthetization of the patient, leading to added risk and cost.

In response to the foregoing concerns, many medical professionals use less invasive procedures, such as endoscopy. During an endoscopic procedure, the physician may make a small incision in the patient's skin, and insert, into the patient, a small optical transmission device, such as a camera, or a specimen collection tool, such as biopsy forceps. Alternatively, the physician may insert the endoscopic devices into the patient's body via a natural pathway, such as the digestive track or mouth. By employing endoscopic procedures, the physician may be able to perform exploratory surgery with minimal discomfort and risk to the patient.

Normal procedures call for sterilizing endoscopes following use, and storing the endoscopes, which are somewhat delicate instruments, for their next use. Moreover, because of the advantages of endoscopic procedures, they may be performed quite frequently at hospitals and clinics. It is not uncommon for hospitals to perform dozens of endoscopic procedures in a relatively short window of time. Unfortunately, hospitals may be limited in the number of endoscopes that are available. That is, the same endoscope may be used on several different patients in the short window of time. Such circulation may require medical professionals to employ certain care protocols with respect to the equipment. For instance, to prevent against the spread of disease, again for example, the endoscope must be cleaned and sterilized between uses. Additionally, frequent circulation of the endoscope, and its components, may entail movement from location to location several times a day, and, as such, may subject the sensitive components of the endoscope to damage.

Ideally, an endoscope, particularly a probe end of the endoscope which may contain sensitive components, is placed into a protective case between uses. That is, the practitioner or his assistant would remove the endoscope from its protective container, use the endoscope, and carefully return it to the container after a thorough cleaning and sterilization. If such protocols are rigidly followed, the sensitive components of the endoscope remain protected when not in use, and the practitioner is quickly able to ascertain which endoscopes were available for use. However, the devices are often not returned to an individual container, and even if they are, they must still be handled and transported for sterilization.

In many health care environments, the endoscope and its components, once used, are placed into a storage area for cleaning. For example, the contaminated portions of the respective endoscopes may be hung on a common railing until the appropriate time at which the endoscopes are collectively cleaned and sterilized. Unfortunately, when so stored, the sensitive components of the endoscopes may contact various objects and even collide with one another, thereby damaging the endoscope. Moreover, because of the frequency with which endoscopes are used, the practitioner may lose track of which endoscopes are ready for use and which require sterilization. Thus, to err on the side of caution, the practitioner may be required to cleanse again those endoscopes for which a usage status cannot be accurately accounted. Additionally, the frequent transportation of exposed endoscope components leaves the sensitive optics, for example, susceptible to damage. Indeed, a fair amount of care must be taken in properly carrying and transporting endoscope components between locations. If damaged, endoscope components can be relatively costly to repair and replace.

The present technique, an exemplary embodiment of which is discussed below, addresses many of the foregoing concerns.

### BRIEF DESCRIPTION OF THE INVENTION

The present technique provides an apparatus for the protection of an endoscope and its components. For example, according to one aspect of the present technique, a cover for use with an endoscope is provided. The cover comprises a body having a recessed portion that is configured to releasably secure to an insertion portion of the endoscope.

According to another aspect, the present technique provides an apparatus for the identification of endoscopes and endoscope components. For example, the present technique may comprise a cover for use with an endoscope in which the cover comprises an indicium configured to indicate a condition or status of the appropriate endoscope or endoscope component.

According to yet another aspect, the present technique provides an endoscope system. The system comprises an endoscope having a light source that produces a light beam, and a flexible conduit coupled to light source that is configured to direct the light beam. Additionally, the system comprises first and second cover members that respectively comprise indicium indicative of first and second endoscope conditions.

The present technique, also provides a method of covering a portion of an endoscope. The method comprises securing a first cover having indicium indicative of an endoscope condition to a portion of the endoscope. The method also comprises removing the first cover from the given portion and placing, onto the portion, a second cover having a second indicium indicative of a second endoscope condition.

Additionally, the present technique offers a method of manufacturing a cover for a portion of an endoscope. The method comprises, shaping a flexible synthetic material to form a cover that is releasably securable to a portion of an endoscope. The method further comprises integrating, with respect to the cover, an indicium indicative of a status of the endoscope.

### BRIEF DESCRIPTION OF THE DRAWINGS

The foregoing and other advantages and features of the invention will become apparent upon reading the following detailed description and upon reference to the drawings in which:

FIG. 1 is an illustration of an exemplary endoscope system incorporating various aspects of the present technique;

FIG. 2 is a perspective view of a cover, in accordance with an exemplary embodiment of the present technique, illustrated in a decoupled configuration in relation to a probe portion of an endoscope, the cover having a drainage hole extending therethrough;

FIG. 3 is a cross-sectional view along line 3-3 of the exemplary cover introduced in FIG. 2;

FIG. 4 is a partial cross-sectional view of an alternate exemplary embodiment of a cover; and

FIG. 5 is a block diagram delineating an exemplary sequence of steps in accordance with various aspects of the present technique.

### DETAILED DESCRIPTION OF SPECIFIC EMBODIMENTS

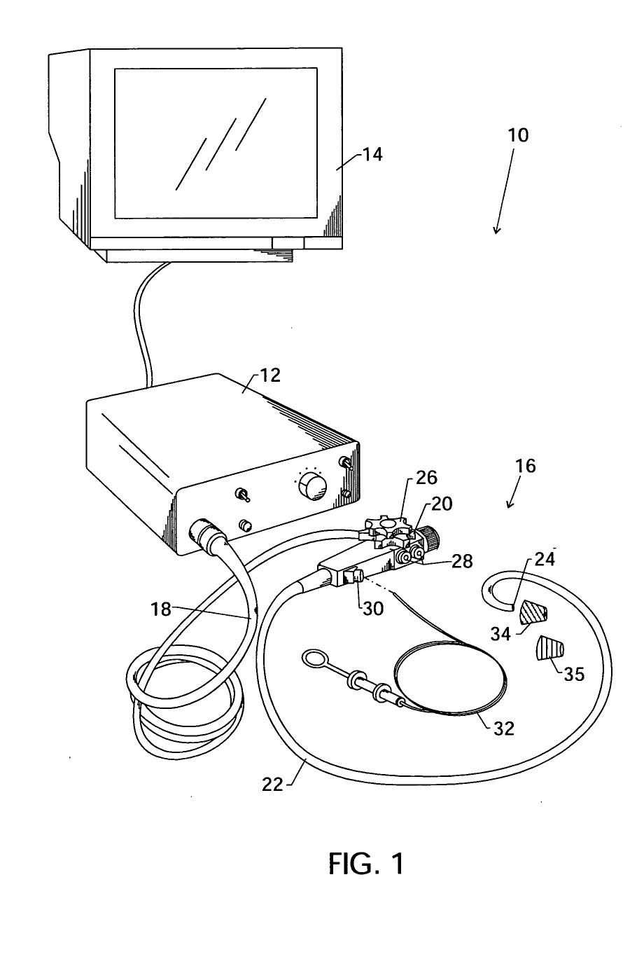

Turning now to the drawings, and referring to FIG. 1, an exemplary endoscope system 10 is illustrated. At the heart of the exemplary endoscope system 10 is a base unit 12 that operates as the nerve center of the system 10. As discussed further below, to display an image signal, the endoscope system 10 may include a display device 14, such as a video monitor, configured to broadcast the images of a patient's internal organs or tissues to a number of medical practitioners simultaneously. Additionally, the system 10 may include an examination wand 16 coupled to the base unit 12 via an umbilical cord 18. Advantageously, the umbilical cord 18 provides a degree of mobility to the examination wand 16. However, if desired, the examination wand 16 may be directly coupled to the base unit 12.

During a procedure, a medical practitioner, such as a physician, may wish to manipulate the examination wand 16 to obtain the appropriate image or retrieve the desired tissue specimen. Accordingly, the examination wand 16 may comprise a control head 20 coupled to an instrument shaft 22. Indeed, the physician may insert the instrument shaft 22 into the patient and control many aspects of the procedure, as discussed further below, by way of the control head 20. By manipulating the control head 20 in cooperation with the instrument shaft 22, the endoscope system 10 may obtain results equivalent to those of traditional exploratory surgery procedures, but in a less invasive manner.

To insert the instrument shaft 22 into the patient, the physician may cut a small incision extending through a portion of the patient's skin or may insert the instrument shaft 22 into the patient via a natural pathway, such as the digestive track. In either event, the endoscopic procedure is considerably less invasive than traditional exploratory surgery. For guidance during insertion, the instrument shaft 22 may comprise angle controls (not shown) that adjust the angle of entry of the distal end of the instrument shaft, that is the end of the instrument shaft leading insertion into and through the patient. More specifically, by manipulating the tension within wires disposed along the length of the instrument shaft 22, the lead direction or insertion angle of the distal end may be adjusted. Advantageously, the flexible nature of the shaft 22, in conjunction with its thin profile, provides an agile instrument capable of maneuvering into various cavities within the patient's body.

Located on the distal end of the instrument shaft 22 may also be an instrumented end or probe portion 24. As discussed in further detail below, the probe portion 24 may contain features advantageous to diagnosing the patient. For example, the probe portion 24 may contain optical components for the receipt and transmission of images as well as specimen retrieval features capable of obtaining tissue specimens for further examination. By guiding the probe portion 24, in certain instances via the images obtained, to the desired location, the physician may be able to diagnose quickly any problem and, accordingly, may also be able to propose a responsive treatment protocol in an expeditious manner. Indeed, real-time visualization of a patient's condition, specifically tissue condition, may be a powerful diagnostic tool.

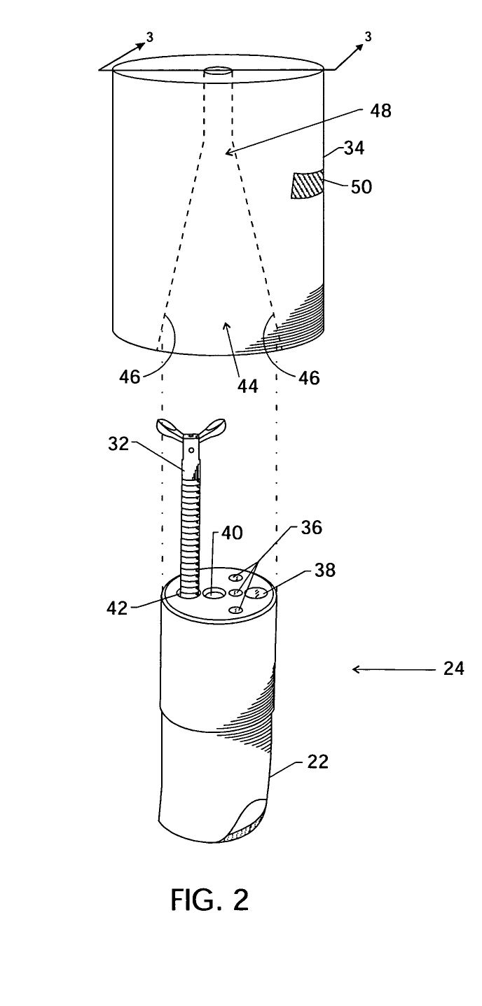

To improve the image quality returned to the base unit 12 as well as to improve the positioning of the probe portion 24, it may be beneficial to flush away excess fluids and tissue along the insertion pathway. Accordingly, the endoscope system 10 may include a mechanism for providing pressurized streams of air and water. In the exemplary system 10, pressurized streams of air and/or water may be provided by the base unit 12, routed through the control head 20, and directed into the patient via the instrument shaft 22. To pressurize the supplied air and water, the base unit 12 may include an integrated compressor unit (not shown). It is worth note, however, that the pressurized air and water may also be provided by a source independent of the base unit 12, such as a wall connection or independent compressor. Once pressurized, air and water may then be routed from the base unit 12, to the examination wand 16 via the umbilical cord 18, and emitted from an aperture (see FIG. 2) in the probe portion 24.

In certain instances, the physician may desire to dynamically manipulate the air and water flow rate. For example, the physician may wish to either increase or decrease the flow rate or pressure of the water or air emitted from the probe portion 24 of the endoscope. Moreover, the physician may wish to selectively engage and interrupt the respective flows. Accordingly, the control head 20 may include flow control valves 26 designed to selectively increase or decrease the flow rate and pressure of the respective air and water streams. For example, by rotating the respective flow control valves 26 in the appropriate directions, the physician may control the amount of water and air emitted by endoscope 10 as well as the pressure thereof. Additionally, the control head 20 may comprise triggers 28 that respectively control, in a binary fashion, the flow of each stream. In other words, when either an air or water stream is desired, the physician may simply depress the respective trigger 28, thereby engaging the appropriate stream. Subsequently, to interrupt the stream, the physician may simply release the trigger 28.

The control head 20 may also comprise a biopsy port 30 that facilitates insertion of various tools, such as biopsy forceps 32, into the patient. During a procedure, as discussed in further detail below, the biopsy forceps 32 may be fed into the biopsy port 30, directed through a conduit within the instrument shaft 22, and extended into the patient from the probe portion 24. Accordingly, once the probe portion 24 is at the desired location (i.e., location of the tissue to be diagnosed), the physician may actuate the appropriately routed forceps and retrieve a tissue specimen from the patient. Advantageously, the tissue specimen may be retrieved in a manner less invasive than traditional exploratory surgery procedures.

After the physician has either removed the desired tissue specimen or viewed the appropriate location within the patient, he may then wish remove the instrument shaft 22 from the patient. Because the instrument shaft 22 has been in intimate contact with the internal tissue of the patient, the instrument shaft 22 must be decontaminated and sterilized prior to its next use. Indeed, the lack of properly maintained instruments may increase the likelihood of disease and bacteria transmission between patients. Accordingly, hospital staff thoroughly clean and sterilize the endoscope and its components prior to the next use.

In many situations, hospital staff may not be able to immediately clean, disinfect and properly store the endoscope for its next use. In typical practice, the examination wand 16 (i.e., the control head 20 and the instrument shaft 22) of an endoscope system 10, once used, is disconnected from the umbilical cord 18 and, subsequently, taken into a storage and cleaning room. In the storage room, the wand may be placed amongst other examination wands 16, some of which may have been previously sterilized. Accordingly, it may be difficult to maintain an appropriate accounting of which wands 16 are clean and which wands 16 are in need of cleaning.

Thus, as discussed further below, to differentiate between those endoscopes that are contaminated and those that are sterilized and prepared for use, the endoscope system 10 may comprise covers 34 and 35 that releasably secure to the probe portion 24 of the instrument shaft 22. For example, the first cover 34 may comprise an indicium, such as a green marking or coloration, intended to indicate a sterile condition of the examination wand 16. In contrast, the second cover 35 may comprise a second indicium, such as a red marking or coloration, intended to indicate a contaminated or used condition of the examination wand 16.

A detail view of the probe portion 24 of the instrument shaft is provided in FIG. 2. It may comprise any number of sensitive components. For example, the probe portion 24 may comprise light beam sources 36. The light beam sources 36 may comprise fiber optic cables extending the length of the instrument shaft 22 that are configured to direct the appropriate light beam produced at the base unit 12 (FIG. 1) and emit these beams from the probe portion 24. Those of ordinary skill in the art will appreciate the use of fiber optics and fiber optic cables as well as the operations thereof. Each light beam source 36 may be configured to emit a respective color or frequency of light. Because video-endoscopes, as discussed with regard to the present embodiment, typically employ RGB monitors, it may be advantageous to emit particular colors of light. Accordingly, each of the light beam sources 36 may be configured to emit one of the three-basic colors: red, green, and blue. By employing these three basic colors, vivid images of the location in need of diagnosis may be produced. However, it is worth note that the three sources may be replaced by a single white light source if so desired. Indeed, any number of colors and light sources are envisaged.

The probe portion 24 may also comprise a lens assembly 38 configured to receive the reflected red, green and blue light beams and convert these into an image signal for transmission to the display monitor 14 (FIG. 1). For example, in the presently described exemplary video-endoscope, the red, green and blue light beams respectively emitted from the beam sources 36 reflect off of the patient's tissue and are received by the lens assembly 38. Behind the lens assembly 38, the probe portion 24 may include a charged couple device (CCD) chip (not shown) that receives the reflected light beams and converts these received beams into image signals. In essence, the exemplary CCD is an array of individual photocells that receives the reflected light beams and produces a signal in proportion to the intensity of the respective light beams received. Moreover, the CCD may also comprise an overlay of primary color strips causing the pixels under a particular strip to be responsive only to certain colors of light. Thus, in operation, the CCD converts the reflected light beams into a video or image signal interpretable by the display device 14. It is of note, however, that the present technique may also be applied to other types of endoscopes, such as fiber-optic endoscopes, the operations and details of which are well-known to those of ordinary skill in the art.

Furthermore, the probe portion 24 of the instrument shaft 22 may also comprise non-optical features. For example, the probe portion 24 may comprise an air and water port 40 from which streams of pressurized air or water may be emitted. Additionally, the probe portion 24 may comprise a biopsy port 42 through which any number of examination tools, such as biopsy forceps 32, cytology brushes, sclerotherapy needles or diathermy snares, may be routed. In other words, the biopsy port 42 may provide a conduit for routing the given examination tool to the appropriate tissue location.

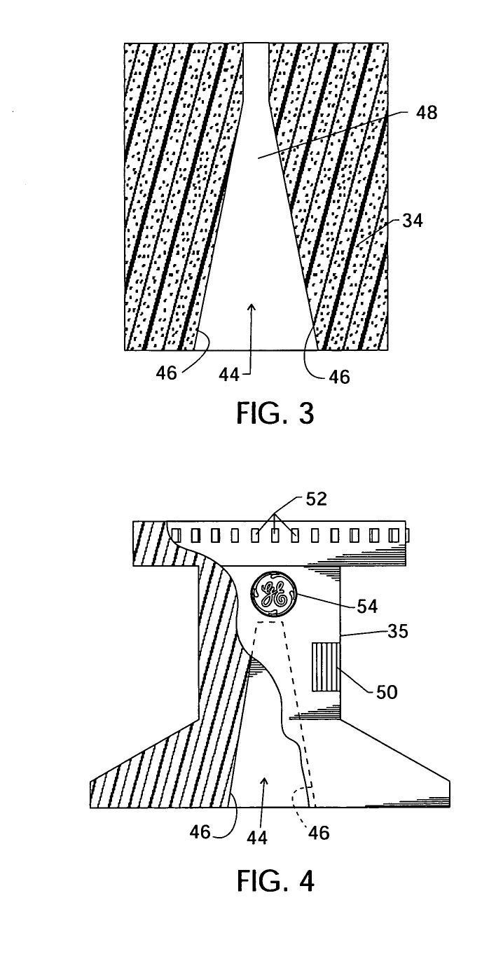

As discussed above, any one of foregoing probe portion 24 features may be sensitive and, as such, susceptible to damage. Additionally, it may be advantageous to provide features that clearly and quickly delineate between sterile and contaminated examination wands 16 (FIG. 1). Accordingly, the exemplary endoscope system may include a cover 34, as illustrated in FIGS. 2 and 3, and as introduced in FIG. 1.

The cover 34 may be a cylindrical or frustroconical shaped body formed of a flexible material, such as plastic or foam, however, multiple combinations and kinds of materials are envisaged. Indeed, in one embodiment, the cover 34 may be formed of an open cell foam. Advantageously, the use of flexible materials provides impact resistance to the cover 34 and, as such, may protect the sensitive features of the probe portion 24 from inadvertent damage. Moreover, the cover 34 may be formed at relatively inexpensive materials, thereby facilitating disposable use of the covers. The cover 34 may also include a recessed portion 44 that extends into the body of the cover. In the exemplary embodiment, the recessed portion 44 may be defined by tapered surfaces 46 that decrease in separation distance as the recessed portion 46 extends into the body. That is, the diameter of the recessed portion 44 decreases as it extends into the body of the cover 34.

To facilitate easy coupling or decoupling of the cover with the probe portion 24, the diameter of the external opening of the recessed portion 44 may be slightly larger than that of the probe portion 24. However, because the diameter of the recessed portion 44 decreases as it extends into the body of the cover 34, the insertion of the cover 34 onto the probe portion 24 is limited. In other words, as the cover 34 is placed onto the probe portion 24, the tapered surfaces 46 begin to engage with the external surfaces of the probe portion 24. Indeed, the elastic and flexible nature of the cover 34 places an inwardly directed grasping force on the probe portion 24. That is, the cover 34 presses inwardly against the probe portion 24 while the probe portion presses outwardly against the cover. This collection of forces working in conjunction with one another provides a sufficient amount of grip to releasably secure the two sections together.

In certain instances, as discussed further below, the cover 34 may be placed onto the probe portion 24 almost immediately after the examination instrument 16 has been cleaned and sterilized. As such, the probe portion 24 may still be moist or wet. To facilitate removal of this moisture, thereby drying the probe portion 24, the cover 34 may include a channel or drainage port 48. The drainage port 48, in the exemplary embodiment, couples to the recessed portion 44 of the cover, and, as such, extends through the body of the cover 34. Advantageously, moisture on the probe portion 24 is directed out through the drainage port 48, thereby allowing the probe portion 24 to drip or air dry. Advantageously, to prevent against the settling of bacteria onto the probe position and to retard the growth of mold due to moisture, the cover 34 may comprise antibacterial and disinfecting agents. For example, the cover 34 may be formed of an antimicrobial plastic, or with an additive provided during or following molding. Moreover, the cover 34 may comprise a coating of disinfectant disposed directly thereon. Indeed, any number of permutations and combinations of disinfectants and antimicrobial agents are envisaged.

Once an examination wand 16, including the probe portion 24, has been cleaned, it may be advantageous to provide one or more indicia indicative of the cover's sterilized status. Accordingly, the cover 34 may comprise an indicium 50, such as a predetermined color, that represents and indicates the examination wand's 16 usage status. For example, to indicate a sterile status, the indicium 50 may comprise a portion of the surface of the cover 34 that is green. In contrast, if the endoscope was in a contaminated state, that is, it has not been cleaned or sterilized since its last use, then the indicium 50 may be red. Such coloration may, of course, be provided in the actual material of which the covers are made. Thus, those aware of the predetermined color-coding system may be able to determine visually and quickly if a certain examination instrument was either ready for use or required sterilization. It is worth note that any number of conditions may be indicated by the indicium. Indeed, if the endoscope is damaged or perhaps in need of maintenance, the cover may comprise an indicium assigned to indicate such a condition.

Moreover, the indicium 50 need not be limited to color. Indeed, the contour or shape of the cover 35 itself may be fashioned so as to indicate the status of the examination instrument to which it is attached. For example, the dumbbell shaped cover 35 of FIG. 4 may indicate to those familiar with the predetermined standard that the examination wand is contaminated and in need of sterilization prior to its next use. Moreover, the contour need not be the overall profile of the covers 34 and 35. Indeed, the indicium may also include raised profile areas 52 predetermined to be indicative of the status of the examination wand. In either event, by simply touching the cover 35, the physician may be able to ascertain the condition of the examination wand or endoscope system. Such touch detectable features may be advantageous in dimly lit areas of a hospital.

Additionally, it should be understood that the indicium 50 may provide other forms of valuable information to the appropriate medical professional. For example, the cover may include indicium that provides information regarding the manufacturer of the endoscope. Indeed, as illustrated in FIG. 4, a stylized trademark or other indicia may quickly inform the user from whom additional information regarding the endoscope may be obtained. Furthermore, the manufacture's indicia 54 may include contact information, such as a telephone number or an e-mail address, identifying whom the medical practitioner may contact to obtain, for example, warranty or technical information.

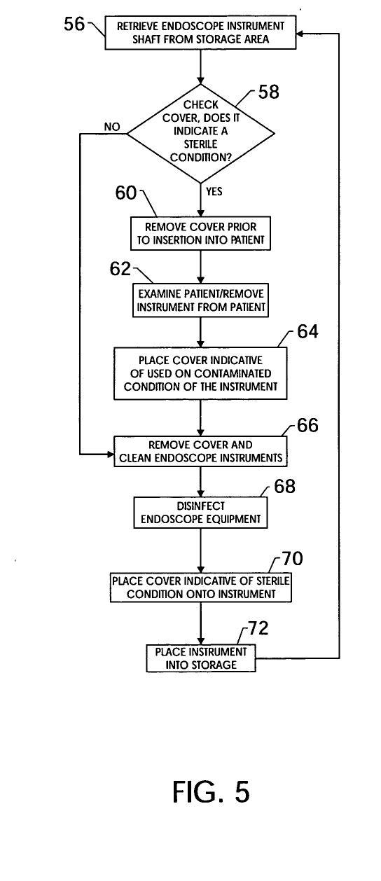

Turning next to FIG. 5 and keeping FIGS. 1 and 2 in mind, an exemplary sequence of steps in operating the present technique is illustrated in flow chart form. Upon deciding that an endoscopic procedure is best for the patient, the practitioner may retrieve the endoscope, more particularly the examination wand 16, from the appropriate storage area, as represented by block 56. Because the storage area may contain a number of examination wands in various conditions, the practitioner may search for an examination wand 16 having an appropriate indicium, as represented by block 58. For example, as discussed above, the practitioner may search for an examination wand 16 having a green indicium 50 on the cover 34 or similar coloration.

Because the green color may be intended to indicate a sterile status of the examination wand 16, the practitioner may be able to determine quickly the number of endoscopes or examination wands 16 that are available for use. Conversely, the practitioner may see a large number of covers 35 each having red colored indicium 50, and, as such, determine that the respective endoscopes and examinations wands 16 must be sterilized and cleaned to keep up with the usage demand.

However, if the indicium 50 is indicative of a sterilized condition, for example green in color, the practitioner may take the appropriate wand 16 from the storage area to the examination room. Advantageously, the impact resistant nature of the cover may protect the sensitive components of the wand 16, as discussed above, from damage. Once the practitioner has transported the wand 16 to the appropriate location, he may then remove the protective cover 34 and insert the wand 16 into the patient, as represented by block 60. If desired, the practitioner may discard the cover into an appropriate waste storage unit.

Upon completing the examination, the practitioner may remove the instrument from the patient, as represented by block 62. As stated above, because the endoscope 10, more particularly the examination wand 16, has been in intimate contact with the patient's tissue, the wand 16 must be cleaned and sterilized prior to its next use. Thus, to indicate that the wand is contaminated, the practitioner may place a cover 35 having indicium 50, such as a red mark, indicative of the wand's contaminated status onto the wand 16, as represented by block 64. Advantageously, as discussed above, the cover 35 may be formed of an impact resistant material, thereby protecting the sensitive components of the wand 16 from damage as the wand 16 is returned to the storage area.

Once a determination has been made that certain endoscopes (i.e., those with the appropriate indicium) need cleaning and sterilization, the practitioner may select these endoscopes for cleaning, and, as such, remove the covers 35, as represented by block 66. Advantageously, the covers 34 and 35 may be discarded, and disposed of in an appropriate waste container. Using appropriate cleaners, such as glutaraldehyde, hydrogen peroxide, peracetic acid, orthophalaldehyde or super-oxidized water, the wand 16 may be sterilized and cleaned for its next use, as represented by block 68.

Subsequent to the cleaning, to both identify the wand as sterilized as well as to protect the sensitive endoscope components, a new cover 34 indicative of the sterile condition may be placed onto the wand 16, and the contaminated cover 35 may be discarded, as represented by block 70. The sterilized wand 16 may then be placed into storage, as represented by block 72, and the entire process repeated over again as necessary.

While the invention may be susceptible to various modifications and alternative forms, specific embodiments have been shown by way of example in the drawings and have been described in detail herein. However, it should be understood that the invention is not intended to be limited to the particular forms disclosed. Rather, the invention is to cover all modifications, equivalents, and alternatives falling within the spirit and scope of the invention as defined by the following appended claims.

1. A cover for use with an endoscope, comprising:
a body having a recessed portion configured to releasably secure to an insertion portion of the endoscope.

2. The cover as recited in claim 1, wherein the recessed portion presents a tapered profile with respect to a longitudinal axis thereof.

3. The cover as recited in claim 1, wherein the body comprises an open cell foam.

4. The cover as recited in claim 1, wherein the body further comprises a channel coupled to the recessed portion such that the channel and recessed portion extend through the body.

5. The cover as recited in claim 1, further comprising an indicium indicative of a condition of the endoscope.

6. The cover as recited in claim 1, further comprising a disinfecting compound disposed thereon.

7. The cover as recited in claim 6, wherein the disinfecting compound is integral to the body.

8. A cover for use with an endoscope, comprising:
a body having a recessed portion configured to releasably secure to a probe portion of the endoscope, wherein the body includes an indicium configured to indicate a condition of the endoscope.

9. The cover as recited in claim 8, wherein the condition comprises a contamination condition.

10. The cover as recited in claim 8, wherein the indicium comprises a predetermined color.

11. The cover as recited in claim 8, wherein the indicium comprises a predetermined contour of the body.

12. The cover as recited in claim 8, wherein the condition comprises an operational condition.

13. The cover as recited in claim 8, wherein the indicium comprises information indicative of the manufacturer of the endoscope.

14. The cover as recited in claim 8, wherein the indicium includes a raised surface with respect to an external surface of the body.

15. An endoscope system comprising:
an endoscope comprising:
a light source configured to produce a light beam; and
a flexible conduit having a probe end and configured to receive the light beam from the light source, wherein the flexible conduit is configured to direct the light beam outwardly with respect to the probe end; and
first and second cover members each having a recessed portion configured to releasably secure to the probe end, wherein the first cover member comprises a first indicium indicative of a first endoscope condition and the second cover member comprises a second indicium indicative of a second endoscope condition.

16. The endoscope system as recited in claim 15, wherein the first and second indicia respectively comprise first and second predetermined colors representative of a sterile endoscope condition and a contaminated endoscope condition.

17. The endoscope system as recited in claim 16, wherein the first color comprises a green and the second color comprises a red.

18. The endoscope system as recited in claim 15, wherein the first and second indicia respectively comprise first and second cover member contours representative of a sterile endoscope condition and a contaminated endoscope condition.

19. The endoscope system as recited in claim 15, wherein the first and second cover members comprise an impact absorbing material.

20. The endoscope system as recited in claim 15, wherein the first and second cover members comprise an open cell foam.

21. The endoscope system as recited in claim 15, wherein the first and second cover members comprise a plastic material.

22. A method of covering a probe portion of an endoscope, comprising:
securing a first cover member having a first indicium indicative of a first endoscope condition to the probe portion;
removing the first cover member from the probe portion; and
securing a second cover member having a second indicium indicative of a second endoscope condition to the probe portion.

23. The method as recited in claim 22, wherein the first and second indicia respectively comprise first and second predetermined colors.

24. The method as recited in claim 22, wherein the first and second indicia respectively comprise predetermined first and second contours.

25. The method as recited in claim 22, further comprising sterilizing the probe portion prior to securing the second cover.

26. The method as recited in claim 22, wherein the first endoscope condition comprises a sterilized condition and the second endoscope condition comprises a contaminated condition.

27. An endoscope system, comprising:
an endoscope including a flexible conduit having a probe portion;
a first means for covering the probe portion, thereby indicating a first status of the endoscope; and
a second means for covering the probe portion thereby indicating a second status of the endoscope.

28. The endoscope system as recited in claim 27, wherein the first endoscope status is a sterilized status and the second endoscope status is a contaminated status.

29. The endoscope system as recited in claim 27, wherein the first and second means respectively comprise first and second predetermined colors.

30. The endoscope system as recited in claim 27, wherein the first and second means are detectable by touch.

31. A method of manufacturing a cover for a probe portion of an endoscope, comprising:
shaping a flexible synthetic material to form a cover configured to releasably secure to the probe portion; and
integrating with respect to the cover an indicium indicative of a status of the endoscope.

32. The method as recited in claim 31, wherein the indicium comprises a predetermined color.

33. The method as recited in claim 31, wherein the indicium is configured to be detectable by touch.

34. The method as recited in claim 31, wherein the flexible synthetic material comprises a plastic.

35. The method as recited in claim 31, wherein the flexible synthetic material comprises foam.

36. An endoscope cover system including an endoscope having an insertion portion, comprising:
a first cover having a first indicia indicative of a used state and a need for sterilization, the cover being configured to receive the insertion portion; and
a second cover having a second indicia indicative of a sterilized state, the second cover being configured to receive the insertion portion.

37. The endoscope cover system as recited in claim 36, wherein the first and second covers comprise an open cell foam.

38. The endoscope cover system as recited in claim 36, wherein the first and second indicia include colors.

39. The endoscope cover system as recited in claim 36, wherein the first and second indicia include contours.

40. The endoscope cover system as recited in claim 36, wherein at least one of the first and second covers is disposable.
